(19) United States
(12) Patent Application Publication
(10) Pub. No.: US 2026/0160749 A1
(43) Pub. Date: Jun. 11, 2026

(54) SYSTEM AND METHOD FOR QUANTIFYING SOIL ORGANIC CARBON USING MULTI-SCALE BIOGEOCHEMICALLY-INFORMED DIGITAL SOIL MAPPING AND UNCERTAINTY ESTIMATION

(71) Applicant: Perennial Climate Inc., Boulder, CO (US)

(72) Inventors: James Kellner, Providence, RI (US); Julia Maddalena, Fort Collins, CO (US); David Schurman, San Francisco, CA (US); Ben Dube, Ithaca, NY (US); Mitchell Donovan, Logan, UT (US); Nick Leach, Squamish (CA); Rose Rustowicz, Longmont, CO (US)

(73) Assignee: Perennial Climate Inc., Boulder, CO (US)

(21) Appl. No.: 19/410,416

(22) Filed: Dec. 5, 2025

Related U.S. Application Data
(60) Provisional application No. 63/729,127, filed on Dec. 6, 2024.

(51) Int. Cl. G01N 33/24

(52) U.S. Cl. CPC G01N 33/245

(57) ABSTRACT

The present invention provides a new and novel method and system for measuring soil organic carbon (SOC) that overcomes the fundamental challenges of accuracy, scalability, and cost. By combining remote sensing, soil samples, machine learning, and biogeochemical principles in a novel way, the present invention achieves highly accurate SOC quantification while dramatically reducing the need for physical soil sampling.

### CROSS-REFERENCE TO RELATED APPLICATIONS

This patent document claims priority to earlier filed U.S. Provisional Patent Application Ser. No. 63/729,127, filed Dec. 6, 2024, the entire contents of which are incorporated herein by reference.

### BACKGROUND OF THE INVENTION

The present invention relates to a system and method for quantifying soil organic carbon (“SOC”) by combining (i) remote sensing data, (ii) soil sample data, (iii) machine learning, and (iv) biogeochemical principles to generate spatially continuous predictions with rigorous statistical validation and uncertainty estimation, enabling accurate measurement of carbon stocks and changes across large agricultural areas while minimizing required soil sampling.

By way of background, the following common terms are known in the industry which are pertinent to the description and disclosure of the present invention. For convenience, these terms and their common definitions are provided below.

As to remote sensing/geospatial terms, the following are described below. 3DEP (3D Elevation Program) is a USGS Digital Elevation Model. BI (Brightness Index) is a remote sensing index associated with total brightness and soil organic matter conditions. EPSG (European Petroleum Survey Group) is a number that uniquely identifies a coordinate reference system. Land Surface Water Index (LSWI) is a remote sensing index that is positively correlated with the total content of liquid water in vegetation and soil. MODIS (Moderate Resolution Imaging Spectroradiometer) is a NASA remote sensing instrument. NBR2 (Normalized Burn Ratio 2) is a remote sensing index that is sensitive to soil moisture and crop residue. NDVI (Normalized Difference Vegetation Index) is remote sensing index that quantifies green vegetation cover. NDTI (Normalized Difference Tillage Index) is a remote sensing index calculated from shortwave infrared reflectance measurements that detects surface roughness associated with tillage status. SAVI Soil Adjusted Vegetation Index) is a remote sensing index that corrects the index in the presence of minimal vegetation cover. SATVI (Soil Adjusted Total Vegetation Index) is a remote sensing index that accounts for both green and dry vegetation while minimizing soil background effects. SAR (Synthetic Aperture Radar) is a form of radar remote sensing that uses airborne or satellite-mounted sensors to create high-resolution measurements of Earth's surface by emitting microwave signals and measuring their reflection, capable of penetrating cloud cover and operating in darkness. SMAP (Soil Moisture Active Passive) is a NASA remote sensing instrument.

As to terms related to soil science, the following are described below. SOC (Soil Organic Carbon) is the carbon component of organic materials in soil, typically expressed as a percentage by mass or as mass per unit area to a specified depth. BD (Bulk Density) is the ratio of the mass of dry soil to its total volume, including pore spaces, typically expressed in grams per cubic centimeter. SOC Stock is the total mass of organic carbon per unit area contained within soils. Digital Soil Mapping (DSM) is the computer-assisted production of maps of soil properties using field observations, laboratory data, and environmental covariates through quantitative relationships. Pedotransfer function is a mathematical relationship used to predict difficult-to-measure soil properties from more easily measured or available soil properties. Variogram/Semi-variogram is a mathematical function describing the degree of spatial dependence of a spatial random field or stochastic process, used to quantify how soil properties vary with distance and direction. Spatial autocorrelation is the degree to which a variable's value at one location is similar to values at neighboring locations, quantified through statistical measures. Temporal covariance is the degree to which a variable's value at one time is similar to values at different times, quantified through statistical measures. Edaphic conditions/variables are properties and characteristics of soil that influence plant growth and organism distribution, including physical, chemical, and biological parameters.

As to statistical and machine learning terms, the following are described below. APE (Absolute Percentage Error) is the absolute value of the difference between predicted and actual values, divided by the actual value and expressed as a percentage. Conformal Predictions (CP) is a framework for generating prediction intervals that satisfy mathematical guarantees of coverage under the assumption that the data are exchangeable. MAPE (Mean Absolute Percentage Error) is the average of absolute percentage errors across multiple predictions. PICP (Prediction Interval Coverage Probability) is the proportion of observations that fall within their predicted intervals, used to validate uncertainty estimates. QRF (Quantile Regression Forests): A machine learning method that extends random forests to estimate conditional quantiles, enabling prediction interval estimation. RMSE (Root Mean Squared Error) is the mean of the squared difference between predicted and actual values. RRMSE (Relative Root Mean Squared Error) is the mean of the squared difference between predicted and actual values divided by the mean of the actual values. XGBoost is a specific implementation of gradient boosted decision trees that uses a more regularized model formalization to control overfitting.

As to methodological terms, the following are described below. CDL (Cropland Data Layer) is a USDA annual raster dataset that contains mapped crop types and other agricultural data. CONUS (Contiguous United States) is the United States of America not including the states of Alaska and Hawaii. Cross-validation is a model validation technique that assesses how well prediction models generalize to independent datasets by partitioning the original sample into training and test sets. Feature engineering is the process of using domain knowledge to extract and combine raw data into features that better represent the underlying problem to predictive models. Fine-tuning is the process of adjusting model parameters and hyperparameters to improve performance for a specific application while maintaining generalizability. GHG (Greenhouse Gas) is greenhouse gases. HWSD2 (Harmonized World Soil Database version 2.0) is a 2023 compilation of national soil surveys and legacy maps distributed as a global 30-arc-second raster. MRV (Monitoring, Reporting, and Verification) is a systematic approach to quantifying and validating environmental parameters, typically used in carbon credit programs. NCEP CFS (National Centers for Environmental Prediction Climate Forecast System) is a 6 hourly summary of weather-related variables at 0.3-degree resolution. POLARIS is a remapping of the SSURGO at 30 m resolution using machine learning and environmental covariates. Prediction Support Unit (PSU) is the defined spatial area and soil volume for which model predictions are calibrated and validated. RaCA (Rapid Carbon Assessment) is a USDA dataset and program to develop estimates of carbon stocks in the United States. SSURGO (Soil Survey Geographic Database) is USDA database that contains soil survey information. USDA is United States Department of Agriculture. USGS is United States Geological Survey. VCM (Voluntary Carbon Market) is a commercial market in which buyers pay for a specified amount net carbon removal and storage, or avoided carbon emissions, for a specified period of time.

In the prior art, by way of background, it is well known that prior to this invention, measuring SOC stocks and stock changes in agricultural lands relied primarily on two approaches, namely, 1) direct soil sampling and 2) biogeochemical process-based modeling. Each method presents significant challenges that limited the scalability and practical implementation of soil carbon projects.

For example, direct soil sampling, while locally accurate, required extensive and costly field operations to collect, transport, and analyze physical soil cores. The spatial variability of soil properties necessitates large numbers of samples to achieve statistical significance, with costs often exceeding $100 per sample. This sampling intensity makes it economically infeasible to accurately quantify SOC across large agricultural areas. Furthermore, the need to resample the same locations over time to measure changes in SOC stocks adds substantial ongoing costs and operational complexity.

To address the shortcomings in these prior methods and systems, biogeochemical process-based models offered an alternative approach by simulating soil processes using physics and chemistry principles. However, these models undesirably require extensive local calibration data and were difficult to deploy at scale, particularly in regions or land use types (e.g. grasslands) without a long history of academic research. They struggle especially with small-holder farms and crop types outside of globally significant crops. Many input parameters required by these models, such as the depth of tillage or the amount of fertilizer applied, are difficult or impossible to measure consistently across large areas and often required unreliable farmer attestations.

However, these biogeochemical process-based models still suffer from various problems and shortcomings. For example, existing publicly available SOC maps and data products, while covering broad geographic areas, demonstrate poor ability to reproduce independently measured values in agricultural lands. These products, including SoilGrids version 2.0, POLARIS, the Harmonized World Soil Database version 2.0, and various 100 m resolution soil property maps, were developed using legacy soil sample data and were not designed specifically for agricultural applications. They exhibited substantial bias in comparison to measured values, making them unsuitable for carbon credit programs requiring accurate quantification.

More specifically, these prior art attempts suffer from many problems and disadvantages. For example, SoilGrids 2.0 uses machine learning for SOC prediction, incorporates multiple environmental covariates, global coverage and validation, and quantifies uncertainty, but it suffers from the disadvantages of relying heavily on legacy soil samples, lacking temporal change detection, and it exhibits poor performance in agricultural lands.

Another known method is the use of biogeochemical Models (e.g., DeNitrification-DeComposition), which incorporate biogeochemical principles, model carbon cycling, predict SOC changes, and have a process-based understanding. However, this prior art attempt requires extensive local parameterization, has no integration with real-time remote sensing, has limited scalability, cannot derive uncertainty from first principles, and has poor performance outside calibrated settings.

There are also commercial providers, such as INDIGO, of Indigo Ag, Inc., that quantify SOC. These commercial providers rely on biogeochemical modeling that is sometimes informed by remote sensing, but such uses are limited in validation data and have no integrated uncertainty framework.

Still further, remote sensing and machine learning had shown promise in other environmental monitoring applications, such as forest carbon quantification, but existing approaches to SOC measurement failed to fully leverage these technologies. Previous attempts to use remote sensing for SOC measurement were limited by the inability to accurately measure subsurface SOC properties, lack of rigorous uncertainty quantification, and suffer from poor validation frameworks. These prior attempts also suffer from insufficient integration of biogeochemical principles and the limited ability to detect changes over time.

Therefore, these limitations create significant barriers to scaling agricultural carbon projects and highlight the need for a more efficient, accurate, and scalable approach to SOC quantification that could support the growing VCM while maintaining scientific rigor.

There is a fundamental challenge and need in soil carbon quantification to achieve accurate, scalable measurement of SOC stocks and their changes over time in diverse agricultural contexts while making such measurements economically viable. However, many needs and problems must be addressed.

First, traditional measurement approaches force an impossible trade-off between accuracy and cost. Direct soil sampling, while accurate at specific points, requires prohibitively expensive and time-consuming physical sampling to achieve statistical significance across large areas. The high spatial variability of soil carbon means that hundreds or thousands of samples may be required for a single project, with each sample costing $100 or more to collect and analyze. This creates an economic barrier that prevents widespread adoption of soil carbon projects. Therefore, there is a need to reduce the cost of soil carbon projects.

Second, existing methods struggle to reliably detect small changes in soil carbon over time. Soil carbon changes gradually, often at rates of 0.5-1% annually. Detecting these subtle changes requires measurement systems with very high precision and well-characterized uncertainty. Prior approaches either lack the sensitivity to detect such changes or require such intensive sampling that measuring change becomes economically infeasible. Therefore, there is a need to reduce the reliance on costly and difficult to use measurement systems.

Third, there is a critical gap in uncertainty quantification and validation that is lacking in many current methods. Previous methods either ignore uncertainty entirely or handle it incompletely, failing to account for crucial factors like spatial autocorrelation and temporal covariance. Without rigorous uncertainty estimation, carbon credit buyers lack confidence in the claimed carbon removals, and carbon credit registries cannot properly assess the quality of credits generated. Therefore, there is a need for better uncertainty quantification and validation.

Fourth, existing approaches to modeling soil carbon lack the sophisticated integration of multiple data sources needed for accurate prediction. Simple remote sensing indices or basic machine learning models fail to capture the complex biogeochemical processes that govern soil carbon dynamics. Meanwhile, complex process-based models require extensive local parameterization that makes them impractical for widespread deployment. Thus, there is a need for better integration of multiple data sources for more accurate prediction results.

The foregoing needs and problems of the industry and the failures of current methodologies create a significant barrier to the generation of accurate spatially continuous predictions with rigorous statistical validity and uncertainty estimation for accurate measurement of carbon stocks.

### SUMMARY OF THE INVENTION

The present invention meets the needs of the industry and solves the problems and shortcomings of prior methods. Therefore, the present invention provides a new and novel solution for providing a comprehensive technical solution for measuring SOC that overcomes the fundamental challenges of accuracy, scalability, and cost. By combining remote sensing, soil samples, machine learning, and biogeochemical principles in a novel way, the method and system of the present invention achieves highly accurate SOC quantification while dramatically reducing the need for physical soil sampling.

In accordance with the present invention, several key innovations are employed. First, the method and system of the present invention provides sophisticated methods for extracting biogeochemically meaningful information from remote sensing data, effectively measuring both inputs to soil carbon (through vegetation indices and biomass estimates) and outputs (through microbial respiration estimates) across entire landscapes. This allows the system to understand and model the carbon cycle at scale, rather than relying solely on point measurements.

Second, the multi-scale approach of the present invention provides unprecedented accuracy in SOC stock estimation. In other words, the present invention provides SOC stock estimation that has a standard deviation of error of the predictions relative to the true value that are much smaller than other methods and systems. At the project (portfolio of fields) level, the method and system of the present invention achieves a standard deviation of the error of the mean of model predictions that is <10% of the true value without any local samples, significantly outperforming existing public SOC maps and traditional measurement approaches. When enhanced with minimal local sampling, the standard deviation of the error of the mean of model predictions decreases to <5% of the true value. This high precision extends to detecting small changes in SOC over time, including changes of the magnitude expected in carbon market programs. This is a significant advance over prior methods and systems.

Third, the present invention provides rigorous uncertainty quantification and validation of estimates through a novel combination of statistical techniques. By employing advanced methods like conformal predictions and quantile regression forests, along with sophisticated handling of spatial autocorrelation, the method and system of the present invention produces statistically valid uncertainty estimates that meet or exceed the stringent requirements VCM programs. It should be understood that other methods for producing prediction intervals and methods of regression may be used and still be considered within the scope of the present invention.

Thus, the practical benefits of the method and system of the present invention are substantial. The system substantially reduces dependence on soil sampling compared to traditional approaches, dramatically lowering project costs while maintaining or improving accuracy. This makes soil carbon projects economically viable across much larger areas which is not possible with prior methods and systems. The continuous nature of the measurements also enables early detection of problems and better project management, while the ability of the system and method of the present invention to work across diverse geographies and agricultural practices makes it broadly applicable.

Importantly, the present invention's validation framework ensures reliability through multiple statistical tests, including coverage requirements, goodness of fit metrics, and bias assessments. This comprehensive validation approach, combined with the system's transparent uncertainty quantification, provides the scientific rigor needed for widespread adoption in carbon markets.

Through these accomplishments, the invention solves the core challenge of making accurate soil carbon measurement both technically feasible and economically viable at scale. This enables agricultural soils to fulfill their potential as a major carbon sink, supporting global efforts to address climate change through carbon removal and storage.

Therefore, the method and system of the present invention provides a comprehensive integration of 1) biogeochemically-informed feature engineering, 2) sophisticated uncertainty quantification and propagation, 3) rigorous validation framework, 4) multi-scale temporal change detection, and 5) proven commercial validation at scale.

Thus, an object of the present invention is to provide a new and novel solution for providing a comprehensive technical solution for measuring SOC that is more accurate than prior art solutions.

Another object of the present invention is to provide a solution that is more scalable than prior solutions.

Yet another object of the present invention is to provide a solution that is less expensive than prior solutions.

A further object of the present invention is that it reduces the amount of soil sampling.

Another object of the present invention is to provide a method and system that produces statistically valid uncertainty estimates.

### BRIEF DESCRIPTION OF THE DRAWING FIGURES

Further advantages, features and possible applications of the present invention are shown and described in the accompanying drawing figures.

FIG. 1 shows a flow chart with supporting graphs and data to show model calibration, validation, and uncertainty estimate flow of the method and system of the present invention;

FIG. 2 shows the generation of predictions of SOC stock in a single field based on a general purpose model and soil core sample locations from a stratified random sample as part of a framework for validation and aggregation of uncertainty in accordance with the method and system of the present invention;

FIG. 3 shows an ensemble of models developed using calibration data in accordance with the framework of the present invention;

FIG. 4 shows the evaluation of PICP on validation data in accordance with the framework of the present invention;

FIG. 5 shows the generation of predictions and uncertainty of SOC (%), BD, and SOC stock in accordance with the framework of the present invention;

FIG. 6 shows the estimation of spatial autocorrelation of the standardized prediction error by generating a variogram in accordance with the framework of the present invention;

FIG. 7 shows the aggregation of uncertainty to the field mean level using Monte Carlo integration of a double integral from Wadoux and Heuvelink equation 7;

FIG. 8 shows that, on average, larger sample sizes result in lower point-level uncertainty further showing uncertainty in digital soil mapping for field-scale carbon stock quantification in accordance with the present invention;

FIG. 9 shows how sample size impacts field-level uncertainty, but with quickly diminishing returns, and how QRF-based models tend to have lower uncertainty than kriging-based models;

FIG. 10 shows a spatial distribution of 5,230 in-situ soil samples used to develop a data-driven model of SOC as a percentage by mass in agricultural soil where the light or darkness of the boxes are proportional to the density of points within each grid cell and where the scale bar is 1,000 km;

FIG. 11 is a table showing the number of physical soil samples collected in combinations of month and calendar year to develop a data-driven model of SOC as a percentage by mass in agricultural soil;

FIG. 12 is a table showing the number of in-situ soil samples in 8 cropland classes in CONUS for each US state to develop a data-driven model of SOC as a percentage by mass in agricultural soil and where crop classification is from the USDA CDL during the calendar year of sample collection;

FIG. 13 is a table showing the number of in-situ soil samples in 8 cropland classes and 10 soil orders in CONUS to develop a data-driven model of SOC as a percentage by mass in agricultural soil and with crop classification from the USDA CDL during the calendar year of sample collection and with soil orders from the USDA-NCSS soil survey data (SSURGO);

FIG. 14 shows 90 covariate features used to predict SOC as a percentage by mass using a gradient-boosted regression tree;

FIG. 15 shows a graph of measured SOC (%) against predicted SOC (%) to show the cross-validated relationship between measured in-situ SOC as a percentage by mass and predicted values from a data-driven model in agricultural soils with the solid line being the 1:1 relationship, and the dashed line being the best-fit linear regression and where this plot shows a random sample of 2,000 points for clarity as part of the full data set that contains 5,230 observations;

FIG. 16 shows five graphs of samples showing relationships between field mean SOC (%) and predicted values where points are 165 fields with ≥5 samples (total number of samples=3,285) with the following data: (A) an instance in accordance with the general approach of the present invention. (B) The 100 m soil properties and class map from Ramcharan et al. (2018). (C) the Harmonized World Soil Database version 2.0. (D) SoilGrids version 2.0. (E) POLARIS using the van Bemmelen factor to convert soil organic matter into SOC. The solid line showing the one-to-one relationship, and the dashed line showing the best-fit linear regression.

FIG. 17 shows MAE between field mean SOC and converted POLARIS SOM using conversion factors over the interval 0-2;

FIG. 18 shows five graphs of samples showing relationships between field mean SOC (%) and predicted values where points are 165 fields with ≥5 samples (total number of samples=3,285) with the following data: (A) an instance in accordance with the general approach of the present invention. (B) The 100 m soil properties and class map from Ramcharan et al. (2018). (C) the Harmonized World Soil Database version 2.0. (D) SoilGrids version 2.0. (E) POLARIS using a soil organic matter to SOC conversion factor of 1.47. The solid line showing the one-to-one relationship, and the dashed line showing the best-fit linear regression.

FIG. 19 is a table showing validation of mean SOC (%) in 165 agricultural fields measured using 3,285 in-situ soil samples where b₀ and b₁ are the intercept and slope of the linear regression relating mean SOC from in-situ soil samples (response) to the predicted value from four publicly available data products and the present invention.

FIG. 20 shows a table of the performance of 20 candidate models used to predict SOC as a percentage by mass using physical soil samples; and

FIG. 21 is a table showing the relative importance for 90 features used to predict SOC (%) in CONUS agricultural soils and the importance is from a gradient-boosted regression fitted to 5,230 measurements of SOC linked to covariate features and summed within feature types where, for example, there are 32 time-series features from Sentinel-2 optical remote sensing whose aggregate importance is 28.2% and totals are marginal sums within feature classes.

### DESCRIPTION OF THE INVENTION

As further shown in the attached figures, the system and method of the present invention is shown and described in detail.

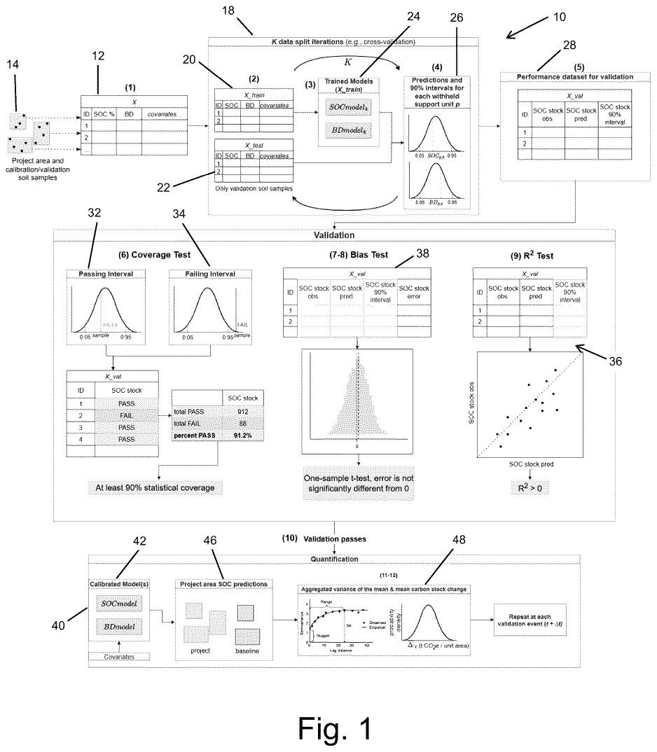

Referring first to FIG. 1, an overview of the method and system 10 of the present invention is shown. Details of the method of the invention are shown in further figures, as will be discussed below. In FIG. 1, a joined data set 12 is created from measured area 14 and soil samples as well as biogeochemically informed remote sensing covariates 16. That data is then used for K data split iterations 18 and cross-validation where a training dataset 20 (X_train) is used to train models 24 SOCmodelₖ and BDmodelₖ. A test dataset 22 and the trained models 24 are used to provide predictions 26 and 90% intervals for each withheld sample p in the test set. Such data is then used to create a performance dataset for validation 28 that includes SOC Stock obs, SOC pred. and SOC Stock 90% interval data. That performance dataset 28 (X_val) is then provided for statistical validation tests for 1) coverage with a passing interval 32 and failing interval 34 for at least 90% statistical coverage, 2) R², where R² is greater than zero as seen in the graph of SOC Stock obs. and SOC stock pred. data. Bias is also determined with a one-sample test as shown in the graph in FIG. 1 where error is not significantly different from 0.

Finally, still referring to FIG. 1, the data from the statistical validation tests are provided for quantification 40 using the calibrated models 42 SOCmodel and BDmodel with Covariates 44. The resultant data is used to generate the SOC predictions 46 for a measured area which is, in turn, used to generate the aggregated variance of the mean 48 as seen in the graph of Semivariance against Lag Distance. The change in SOC over time is computed using the difference between mean estimates at each point time and the variance of the change is calculated using the variance of the mean at each time and accounting for temporal covariance. As a result the SOC is quantified in accordance with the present invention.

The method and system of the present invention is shown. In general, the present invention employs a sophisticated multi-component system that combines (i) remote sensing data processing, (ii) machine learning, and (iii) statistical validation in a new and novel way to accurately quantify SOC. Details of the method and system of the present invention are outlined below.

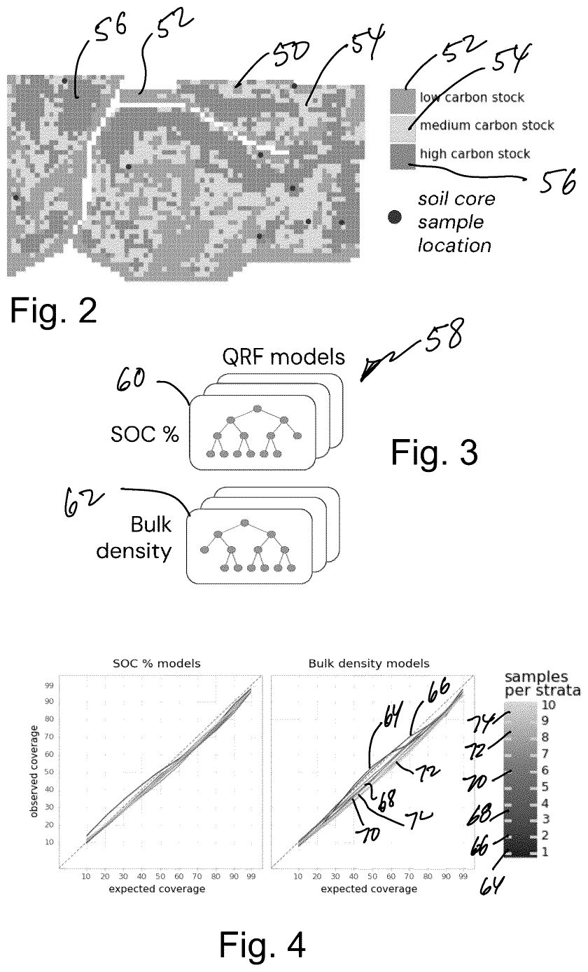

Referring now to FIGS. 2-7, the framework for the generation, validation, and aggregation of uncertainty for a single field in accordance with the method and system of the present invention is shown.

The framework for estimation and validation of the uncertainty of DSM models at different scales in the presence of soil cores 50 collected via a strategic sampling design as in FIG. 2. FIG. 2 shows the generation of predictions of SOC stock 50 in a single field based on a general purpose model and soil core sample locations from a stratified random sample as part of a framework for validation and aggregation of uncertainty in accordance with the method and system of the present invention. This shows a single iteration of three soil cores 52, 54, 56. For this analysis, sampling locations are limited to those where soil cores have already been collected. The image of FIG. 2 shows various soil core sample locations, namely, various soil core sample locations, namely, those with low carbon stock 52, medium carbon stock 54, and high carbon stock 56.

In FIG. 3, after soil cores are collected, QRF 58 are used to estimate uncertainty at the point level for SOC (%) 60 and BD (g/cm3) 62 using independent models. FIG. 3 shows an ensemble of models developed using calibration data in accordance with the framework of the present invention. The SOC percentage 60 and BD models 62 are fit for each iteration of the ensemble.

The data is then validated for coverage on validation data via PICP plots, as in FIG. 4. FIG. 4 shows the evaluation of PICP on validation data in accordance with the framework of the present invention. The average of all iterations for each sample size are shown (by way of example 64, 66, 68, 70, 72, 74), demonstrating coverage is met regardless of sample size.

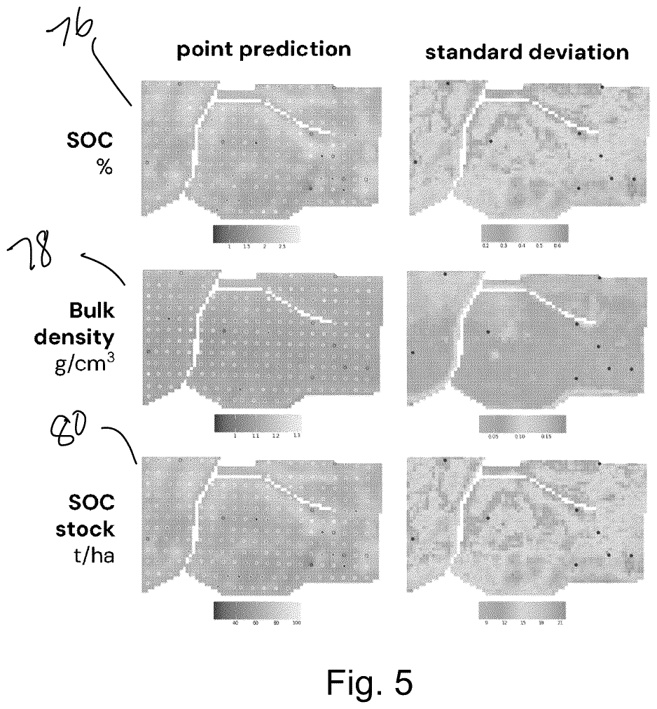

Estimates of uncertainty of SOC stock (t/ha) are then generated, accounting for the correlation between SOC (%) and BD 75 as in FIG. 5, which shows the generation of predictions and uncertainty of SOC (%), BD 78, and SOC stock 80 in accordance with the framework of the present invention. Here, SOC stock is defined as follows:

$$SOC Stock = SOC \% \times BD \times depth ( 30 cm )$$

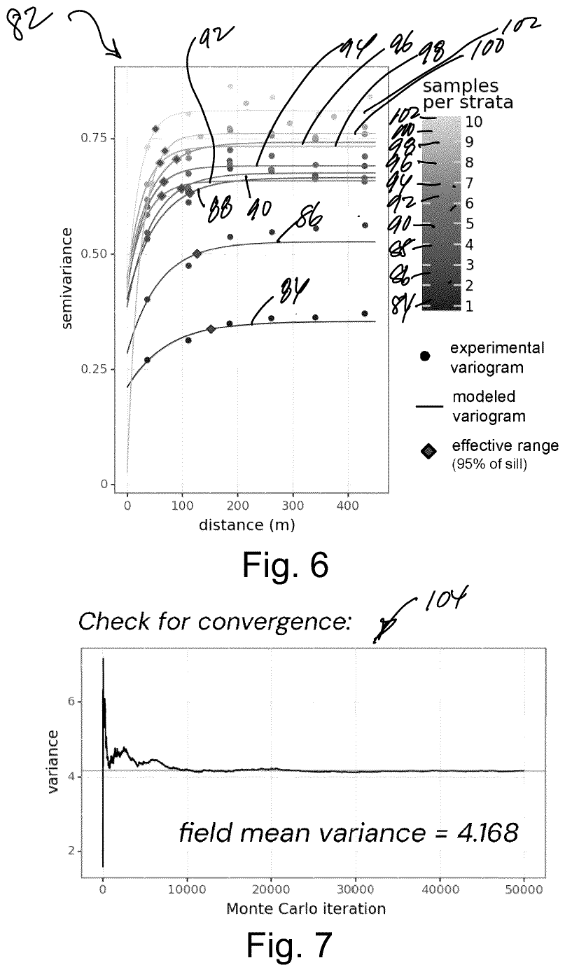

In FIG. 6, a Wadoux and Heuvelink methodology 82 is used for aggregation of uncertainty to the field and field collection level, accounting for spatial

autocorrelation of prediction error. Thus, FIG. 6 shows the estimation of spatial autocorrelation of the standardized prediction error by generating a variogram in accordance with the framework of the present invention where all sample sizes (84, 86, 88, 90, 92, 94, 96, 98, 100, 102) within a single iteration are shown, revealing a reduction of spatial autocorrelation range when more local soil cores are included in training. For example, variograms are fit using all 225 soil cores available.

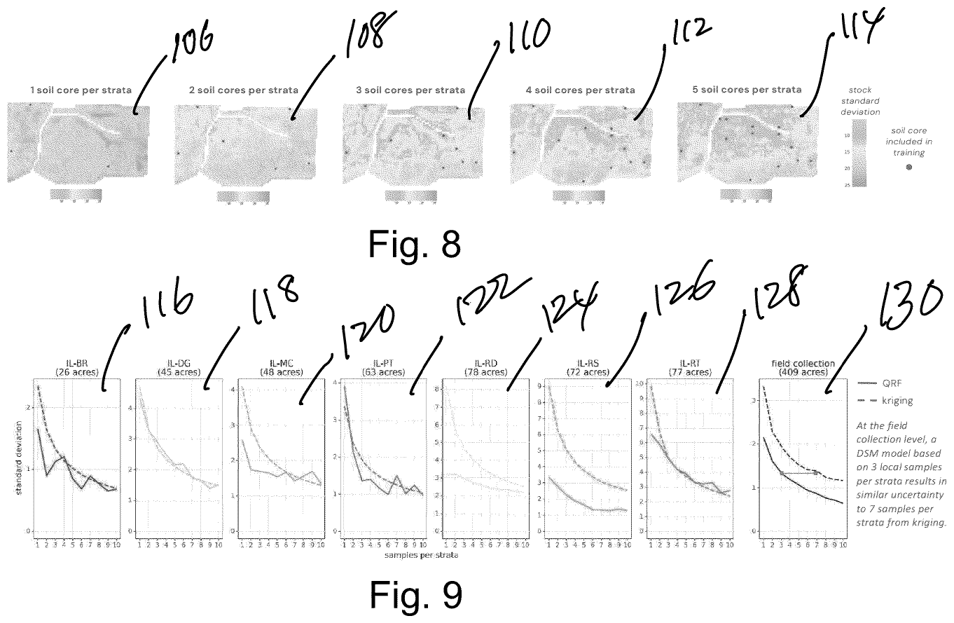

In order to demonstrate how the framework can be applied, publicly available data is used, which includes seven fields in Illinois sampled densely for SOC (%) and BD between 2019 and 2021. In addition to demonstrating the framework for a single field, the impact of sample size on the drivers of uncertainty at the aggregate (field or field collection) level is examined, as in FIG. 7, which shows the aggregation of uncertainty to the field mean level using Monte Carlo integration of a double integral from Wadoux and Heuvelink equation 7, as shown below:

$$\begin{matrix}var ( \frac{1}{{\vert B \vert}^{2}} \int \text{?} ε ( s ) ds ) = \frac{1}{{\vert B \vert}^{2}} \int \text{?} \int \text{?} cov ( ε ( s ) , ε ( u ) ) dsdu \\ = \frac{1}{{\vert B \vert}^{2}} \int \text{?} \int \text{?} σ ( s ) \cdot σ ( u ) \cdot p ( s \vert s - u \vert ) dsdu\end{matrix}$$

$$\text{?} \text{indicates text missing or illegible when filed}$$

Therefore, the standard deviation of SOC stock has decreased from an average of 14.990 t/ha at the point level to 2.042 t/ha at the field level for this iteration of the model.

Because random samples and model stochasticity introduce discrepancies in model results, we conducted 10 executions of stratified random sampling for each sample size. This allows an averaging of the results across all iterations to remove noise extraneous to sample size.

FIG. 8 shows that, on average, larger sample sizes 106, 108, 110, 112, 114 result in lower point-level uncertainty further showing uncertainty in DSM for field-scale carbon stock quantification in accordance with the present invention;

FIG. 9 shows how sample size 116, 118, 120, 122, 124, 126, 128, 120 impacts field-level uncertainty, but with quickly diminishing returns. FIG. 9 also shows how QRF-based models tend to have lower uncertainty than kriging-based models. Estimates of aggregate uncertainty are compared to that of ordinary block kriging in order to ascertain both the correctness of the QRF-based uncertainty estimates as well as whether or not the use of DSM models adds statistical value beyond samples alone.

Referring back to FIG. 6, the sample size impacts field mean uncertainty not only due to the reduction of point-level uncertainty but also due to a decrease in the effective range of spatial autocorrelation of the prediction error. In comparing the QRF-based uncertainty to that of ordinary block kriging, we consistently observe lower uncertainty in the QRF approach, especially at smaller sample sizes. This provides evidence that DSM solutions and their ability to account for ancillary information at every pixel adds value above and beyond samples alone.

The inability to perfectly quantify the correlation between SOC (%) and BD as well as the spatial autocorrelation of the prediction error introduce two sources of uncertainty which may be expanded to include in the framework of the present invention.

Therefore, demonstrating how to estimate uncertainty in carbon stock is a significant advance in the industry. Therefore, the present invention provides a framework for estimating and validating uncertainty at the point level for SOC (%) and BD, as well estimating uncertainty of SOC stock at the field and field collection level. This framework lays a foundation for the rigorous quantification of uncertainty required to generate high quality carbon credits in accordance with the present invention.

As part of the method and system of the present invention, advanced remote sensing preprocessing is employed to extract biogeochemically meaningful information. Rather than using exclusively remote sensing measurements from a single point in time, it develops complex temporal signatures that capture crucial aspects of the carbon cycle. The system processes data from multiple satellite sources, such as Sentinel-2 optical data and Sentinel-1 radar, through specialized algorithms that account for factors like cloud cover, atmospheric effects, and seasonal variations in planting. It generates time-series features that track (a) vegetation coverage, (b) crop residues, (c) soil exposure, and other indicators across multiple growing seasons. These features are engineered specifically to detect agricultural land management practices that influence soil carbon, such as cover cropping, tillage intensity, and crop rotations.

The core prediction engine of the present invention uses a sophisticated machine learning pipeline that employs gradient-boosted regression trees (specifically XGBoost). This architecture is employed for its ability to handle non-linear relationships and complex interactions between features. The system of the present invention treats depth as a direct model covariate rather than relying on traditional surface-to-subsurface relationships, allowing it to dynamically learn how soil carbon varies with depth across different conditions. The model is trained on a combination of proprietary soil samples and public data, with careful attention to maintaining independence between training and validation sets.

A particularly new and novel feature and innovation of the present invention is its uncertainty quantification and validation framework. Expanding on the discussion above, this framework employs two complementary approaches: (i) Conformal Predictions (CP) and (ii) QRF. QRF is used to produce maps of SOC, and CP is used to produce prediction intervals for every pixel value. Together these methods generate statistically valid prediction intervals for each pixel-level estimate. The system then uses advanced geostatistical techniques to aggregate these uncertainties across space, properly accounting for spatial autocorrelation through variogram analysis. This ensures that uncertainty estimates remain valid when scaling from individual points to field and project levels.

More specifically, the validation framework of the present invention implements three rigorous statistical tests: 1) coverage (≥90% of validation observations must fall within 90% prediction intervals), 2) goodness of fit (R²>0), and 3) lack of bias (validated through t-tests). This comprehensive validation ensures the predictions of the method and system of the present invention are reliable and well-calibrated. The validation process uses geographically dependent cross-validation to ensure the model performs well on new locations.

For temporal analysis, the present invention tracks changes in SOC stocks while accounting for the covariance between measurements at different times. It uses specialized preprocessing to ensure temporal consistency in feature generation and employs statistical methods to handle temporal autocorrelation. This enables reliable detection of small SOC changes when aggregated over a carbon market program area.

The method and system of the present invention includes a sophisticated data pipeline built on cloud infrastructure that ensures consistent feature generation between training and inference. This pipeline manages everything from raw data ingestion through feature engineering to final prediction and uncertainty estimation. It maintains version control of models and features while tracking all processing steps for reproducibility.

As noted above, when local samples are available, the system can be further enhanced through model localization. This process fine-tunes the predictions to local conditions while maintaining the statistical validity of the uncertainty estimates. The system determines the optimal weight to give local samples versus the base model by maximizing accuracy while preventing overfitting.

The following additional processes are also employed so the method and system of the present invention may be carried out.

Remote Sensing Preprocessing & Feature Engineering provides for temporal aggregation and filtering with 1) cloud masking and atmospheric correction, 2) seasonal vegetation coverage calculations, and 3) multi-temporal observations targeting key phenological stages. Further, a Spectral Indices Calculation is employed:

$$NDVI = ( NI R - red ) / ( NI R + red ) )$$

$$NDTI = ( SWIR 1 - SWIR 2 ) / ( SWIR 1 + S WIR 2 )$$

$$SATVI = 1.5 \times ( SWIR 1 - red ) / ( SWIR 1 + red + 0.5 ) - ( SWIR 2 / 2 )$$

$$… etc .$$

Also, a Core ML Pipeline Input Layer is used with 1) Remote sensing features, 2) Environmental covariates, 3) Depth as continuous variable, 4) Weather variables, and 5) Soil properties.

To summarize above, the model architecture uses XGBoost as its primary model, QRF for uncertainty as its secondary model, and Conformal predictions for improved prediction intervals as its tertiary model. Uncertainty Quantification Framework Pixel-level uncertainty uses prediction intervals from Conformal predictions or QRF.

Spatial aggregation is defined by the following:

$$Variogram calculation : ρ ( h ) = \frac{s i l l - γ ( h )}{sill}$$

### Covariance:

$$cov ( {σ}_{s , l} , {σ}_{u , l} ) = {σ}_{s , l} \times {σ}_{u , l} \times ρ ( \vert {s}_{l} - {u}_{l} \vert )$$

where the variogram calculation produces a statistically-valid estimate of the spatial autocorrelation in model prediction errors between locations s and u for every Monte Carlo draw 1. This variogram allows the present invention to account for spatial autocorrelation when calculating the area-based estimate of uncertainty (e.g., the project-level variance).

Project-level variance is defined by the following:

$$var ( ) = \frac{1}{L} \times {\sum }_{l = 1}^{L} {σ}_{s , l} \times {σ}_{u , l} \times ρ ( \vert {s}_{l} , {u}_{l} \vert )$$

The project-level variance is an estimate of variability for an area (the project) at time t that accounts for spatial correlation in model prediction errors. L is the number of Monte Carlo draws.

Validation Framework Statistical Tests provide:
Coverage: ≥90% within 90% prediction intervals

$${R}^{2} > 0$$

Bias t-Test Coverage ensures that the 90% uncertainty intervals associated with model predictions contain the true value ≥90% of the time. R²>0 ensures that the model has predictive power above and beyond using samples alone. A bias t test ensures that model predictions nether overestimate nor underestimate true values on average.

The Change Detection System Stock Change Calculation is defined by:

$$Δ {{\_}^{}}_{t , Δ t} = Δ {{\_}^{}}_{t , Δ t} - Δ {{\_}^{}}_{t}$$

The Change Uncertainty is defined by:

$$var ( Δ {{\_}^{}}_{t , Δ t} ) = var ( {{\_}^{}}_{t , Δ t} ) + var ( {{\_}^{}}_{t} ) - 2 {ρ}_{t + Δ t} \times \sqrt{var ( {{\_}^{}}_{t , Δ t} )} \times \sqrt{var ( {{\_}^{}}_{t} )}$$

The variable ρ_{t+Δt} is the correlation of the prediction error of SOC stock between times t and t+Δt. In view of the above, as to remote sensing and feature engineering, the prior art merely provides simple vegetation indices (NDVI, EVI), point-in-time measurement, surface-only measurements and a small number of data sources and predictors, while the present invention provides biogeochemically-informed feature engineering targeting carbon cycle components, multi-temporal phenological signatures for practice detection, depth as a continuous model covariate as well as integration of temporally-aligned environmental and remote sensing variables.

As to machine learning architecture, the prior art merely provides single model approaches, fixed prediction intervals, and independent pixel predictions while the present invention provides the improved features of multi-model ensemble combining XGBoost with QRF/CP for uncertainty, dynamic prediction intervals based on local conditions, and spatial dependency handling through variogram analysis.

As to uncertainty quantification, prior art methods employ simple error metrics (RMSE, MAE), independent error assumption, point-based uncertainty, and fixed uncertainty bands. The present invention provides improved uncertainty quantification by using dual uncertainty quantification (CP and QRF), explicit spatial autocorrelation modeling, cross-scale uncertainty propagation methodology, and temporal covariance incorporation.

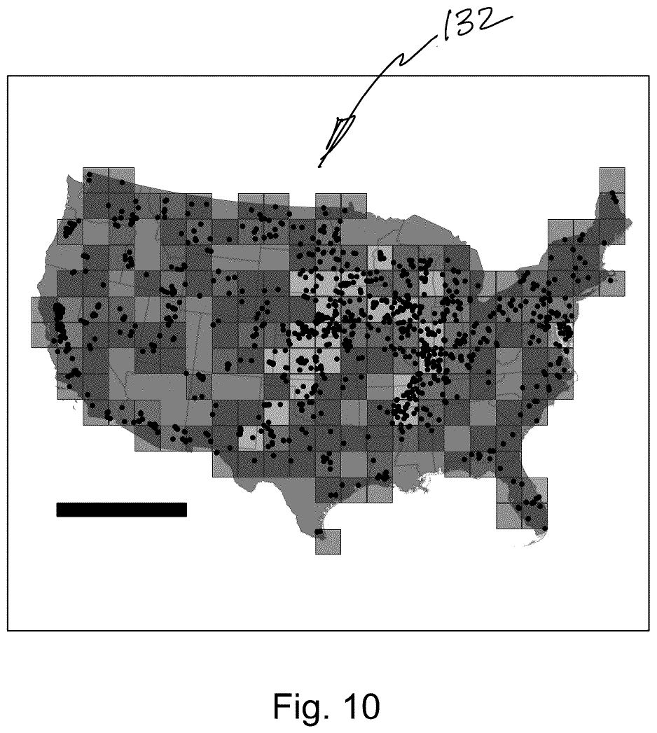

Referring now to FIGS. 10-21 further details and sample data and sample scenarios are provided to further illustrate the method and system of the present invention and how the data is processed to provide highly accurate SOC quantification and uncertainty estimation.

As discussed above, carbon market programs in agriculture depend on accurate measurements of SOC that can be deployed at scale efficiently, but barriers are preventing widespread adoption. To overcome these challenges, the present invention provides a DSM framework driven by machine-learning and numerous spatial covariates, including long-term climate proxies, short-term climate and weather-related variables, topographic and edaphic measurements, and remote sensing time-series summaries. Discussed below is a specific example to show that the model can predict SOC (%) in the top 30 cm of soil using 5,230 measurements of SOC (%) in agricultural land within 47 states in CONUS. Model predictions closely matched independent measured values. The intercept and slope of the cross-validated relationship at the agricultural field level were −0.179 and 1.095. The coefficient of determination was R²=0.811, and the RRMSE was 0.041. In contrast, comparison of independent field measurements to four publicly available SOC data products using 165 fields that contained 3,285 in-situ soil samples showed poor ability of existing public SOC maps to reproduce measured values, underscoring the importance of quantification technologies developed specifically for agricultural land and with recent soil measurements. Three prior SOC data products underestimated SOC (%) at small values and overestimated it at large ones, while one underestimated SOC (%) at all values examined. Analysis of feature importance showed that time series summaries from Sentinel-2 are the strongest predictors, followed by temperature variables and features related to surface hydrology. These findings underscore the value of geographically representative training and validation data for quantifying SOC (%) in agricultural land and demonstrate that feature engineering can increase the sensitivity of SOC quantification to optical remote sensing summaries. Data-driven algorithms can generate accurate estimates of field-level SOC (%) in agricultural land in CONUS that overcome barriers to scale in carbon market programs.

SOC plays an important role in carbon market programs due to its ability to mitigate and offset GHG emissions through implementation of regenerative agricultural practices. Activities such as cover cropping, reduced tillage and changes to crop rotation increase SOC storage and remove GHG from the atmosphere. The VCM incentivizes these practices by paying for GHG reductions and removals, which can be used to offset emissions elsewhere or sold on a secondary market.

A key challenge to the development of a robust VCM in agricultural soils is quantification. Current methods are uncertain and expensive to scale. Direct measurement, such as in-situ soil sampling, is locally accurate and can be extrapolated to larger regions but may require large sample sizes to overcome spatial variation. An alternative to direct measurement is biogeochemical simulation. Simulation models forecast SOC sequestration using physical, chemical, and biological principles, but they are difficult to deploy at scale and in regions without a long history of academic research, including small-holder farms and crop types other than globally significant crops.

DSM is able to overcome these measurement challenges. DSM refers to the practice of using empirical statistical and machine learning models to predict SOC (%) by associating in-situ soil samples with spatial covariates, including climate proxies, weather, soils, topography, and remote sensing data. This approach is similar to methods used to estimate aboveground forest carbon in carbon market programs. Numerous studies have shown that environmental and remote sensing variables can map SOC in soils.

Two criteria necessary for the success of carbon market programs in agricultural soils are the accuracy of area-based summaries and sensitivity of predictions to variables related to on-the-ground farm practice. Carbon market programs credit SOC sequestration within areas that can be hundreds of hectares or larger, requiring measurement techniques to be validated at the unit of agricultural land management, which may be the individual field, ranch, or collections of management units that include hundreds to thousands of individual parcels. Sensitivity to management practices is necessary to ensure that changes in SOC are attributable to changes in land management, which is a requirement of carbon market programs.

In this example, the Advanced Terrestrial machine-Learning Analysis System for Soil Organic Carbon (ATLAS-SOC) is developed. ATLAS-SOC is a data-driven DSM framework to quantify SOC in the top 30 cm of soil in non-tree crops in CONUS for use in carbon market programs. Time-series features increase the sensitivity of remote sensing variables to SOC prediction using high-resolution satellite data from Sentinel-2 and other environmental covariates. Model performance is evaluated with a geographically dependent cross-validation at both sample and field levels and benchmarked against existing publicly available SOC data products. Thus, as noted above, publicly available SOC data products perform poorly when validated against recent in-situ soil samples, whereas ATLAS-SOC produces accurate estimates of field-level SOC, underscoring the potential of data-driven SOC quantification in support of robust carbon market programs in agricultural soils.

Data-driven calculation of SOC depends on the relationship between independently measured variables and SOC from in-situ soil samples. SOC (%) is the amount of SOC in a given soil sample expressed as a percentage of oven-dry mass. The approach developed here allows in-situ soil samples collected at different locations, depths below the surface, and times to be used for model calibration and validation. It does this by using locally measured covariates generated at times close to sample collection, and by treating depth as a continuous covariate feature. This indicates that sample data is always associated with covariate features that coincide in space and time, even though samples themselves have been collected at different times.

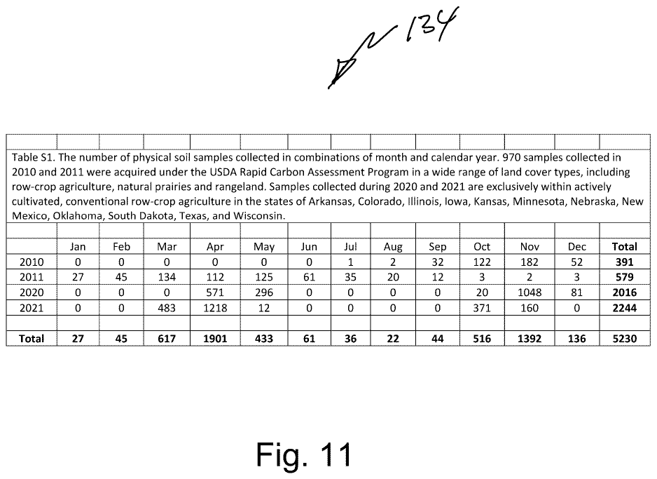

In an example, as shown in FIG. 10, the method and system of the present invention preferably uses 5,230 in-situ soil samples collected within 410 agricultural fields where the spatial distribution of the 5,230 in-situ soil samples are used to develop a data-driven model of SOC (%) in agricultural soil. The shades of the boxes are proportional to the density of points within each grid cell. Scale bar is 1,000 km. In FIG. 11, the number of physical soil samples 134 collected in combinations of month and calendar year. 970 samples collected in 2010 and 2011 were acquired under the USDA Rapid Carbon Assessment Program in a wide range of land cover types, including row-crop agriculture, natural prairies and rangeland. Samples collected during 2020 and 2021 are exclusively within actively cultivated, conventional agriculture in the states of Arkansas, Colorado, Illinois, Iowa, Kansas, Minnesota, Nebraska, New Mexico, Oklahoma, South Dakota, Texas, and Wisconsin.

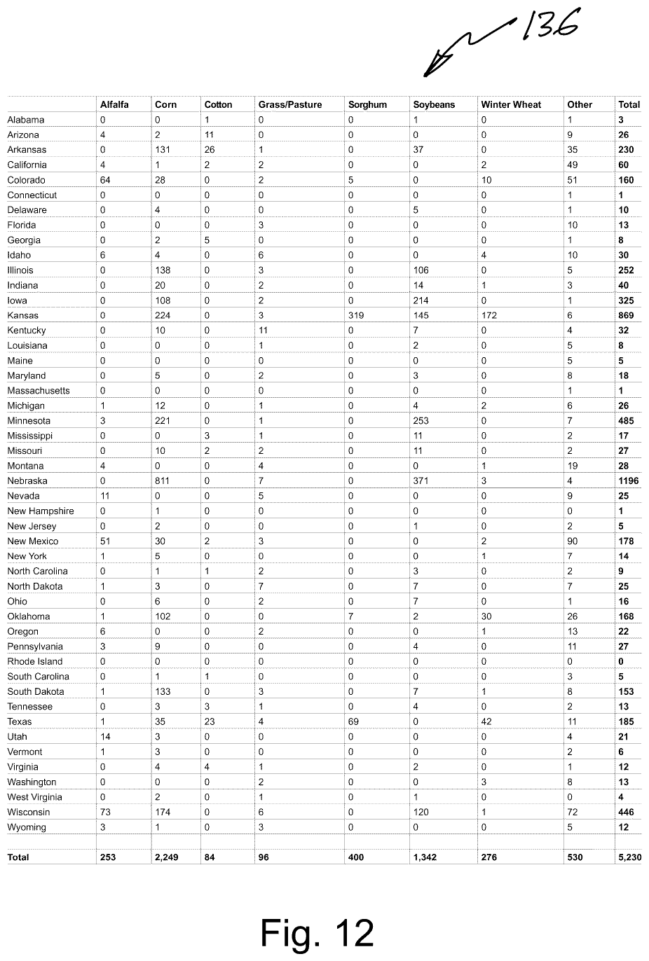

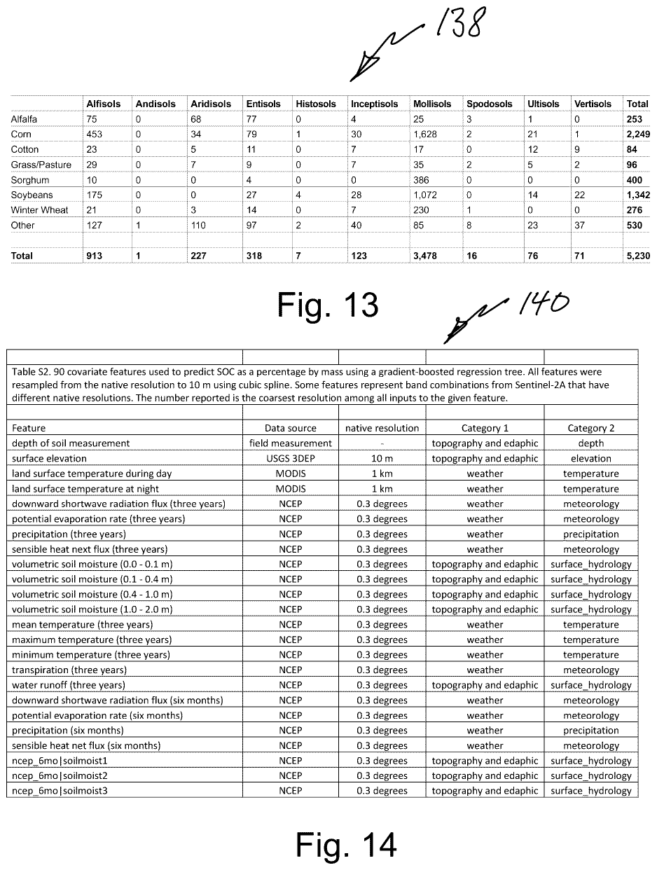

In FIG. 12, a table 136 shows the number of in-situ soil samples in 8 cropland classes in CONUS where the crop classification are from the USDA CDL during the calendar year of sample collection. Most of these samples (4,260) were collected within actively cultivated, conventional row-crop agriculture in the states of Arkansas, Colorado, Illinois, Iowa, Kansas, Minnesota, Nebraska, New Mexico, Oklahoma, South Dakota, Texas, and Wisconsin during the years 2020 and 2021. The remaining 970 samples were collected in 2010 and 2011 under the USDA Rapid Carbon Assessment (RaCA) program. At the time of collection, samples represented corn (2,249), soybeans (1,342), sorghum (400), and alfalfa (253), with the remaining 1,239 samples among less common conditions with <100 samples each (e.g., grassland and pasture, cotton, spring wheat, and rice, which are shown at 138 in FIG. 13. Crop types were identified using the USDA CDL associated with the calendar year of sample collection. Measurements in 2020 and 2021 were acquired at randomly selected locations in fields that could be accessed before the growing season or after harvest, subject to the constraint that no sample location was within 5 m of a field boundary. The data set consists of 4,168 measurements in the 0-5 cm depth range, and 1,062 measurements of 0-30 cm. All samples were shipped to an analytical laboratory where SOC (%) was determined using the method of dry combustion. Most samples represent Mollisol soils (3,478), followed by Alfisols (913), Entisols (318), Aridosols (227) and Inceptisols (123). Also shown in FIG. 13 is a table that shows the remaining samples are among soil orders with <100 samples each. Such soil orders were identified using the USDA NRCS SSURGO.

Predictors include features related to climate, topography, and biophysically meaningful variables derived from optical remote sensing that are correlated with soil properties and formation. In accordance with the present invention, 90 features are identified using data products related to climate and weather, soil properties and topography, land use, vegetation, management, and other characteristics, as seen in the table 140 in FIG. 14. These 90 covariate features are used to predict SOC (%) using a gradient-boosted regression tree where all features were resampled from the native resolution to 10 m using a cubic spline. Some features represent band combinations from Sentinel-2A that have different native resolutions. The number reported is the coarsest resolution among all inputs to the given feature.

This demonstrates that combinations of long-term climate variables, topography and optical remote sensing are predictive of organic carbon content in soils. Because our analysis addresses agricultural land in particular and was designed to evaluate whether SOC (%) in agricultural soils can be predicted using DSM methods, we focused on the selection of covariate features known to be associated with SOC (%) through biological, geological, or farm-practice relationships.

As to covariate data using the concept of resolution, resolution is equivalent to pixel size, where native resolution refers to the source data product before reprojection or sampling, and fundamental resolution is the scale at which the feature varies in the real world. For example, the mean annual temperature from WorldClim may have a fundamental resolution of tens of kilometers, even though it is gridded at 30 arcseconds (about 1 km). Although covariate features to 10 m resolution are resampled using a cubic spline or nearest-neighbor procedure, resampling does not change fundamental resolution. Differences in fundamental resolution among covariate features are characteristics of the environment that are consistent with the state-factor framework in the context of DSM. Other data-driven approaches to carbon quantification also use features at varying fundamental and native resolution. Hierarchical variation in fundamental resolution among features is a well-defined characteristic of data-driven analysis of environmental properties.

In accordance with the present invention, values are extracted from covariate data sets at the location of each sample point. The covariate data set includes upper and lower points of soil sample measurement as quantitative predictors. This allows the algorithm to use measurements collected at different depths in the soil profile to produce standardized output over the 0-30 cm depth range. For the features described below, all features are assigned to SOC measurements acquired in 2020 and 2021. Data collected under the USDA RaCA program in 2010 and 2011 precede the availability of Sentinel-1 SAR, Sentinel-2 bottom of atmosphere surface reflectance, and SMAP data products. For samples acquired prior to 2020, these covariates are treated as missing values.

Missing values occur when no observation is possible or due to spatial or temporal gaps in the data record. This can occur when cloud cover results in data loss. For example, the following simplified feature set x=[1, 3, NA, 5] is used. This feature set represents four covariate features that could be associated with a single response. The first, second and fourth features have numerical values, but the third feature is missing, indicated by NA. One solution to missingness is to eliminate records that contain at least one missing feature value, called complete-case analysis. This is not ideal, because many records contain at least one missing value, resulting in large data losses. An alternative approach to dealing with missing data imputes missing observations using a predictive model and then analyzes the combined set of complete cases and imputed values using the same methods that are applied in a complete-case analysis. Here a third approach developed in the context of gradient-boosted regression trees is used according to the present invention. The approach identifies the default direction of the split in the decision tree for cases where the required feature is missing using non-missing records for that feature and then selects the default direction.

Physical-climate proxies include the WorldClim bioclimatic variables, which contain summaries of mean annual temperature and precipitation, temperature and precipitation seasonality and extremes at 30 arcsecond resolution. These variables represent conditions in the 1970-2000 time interval and therefore define typical climate characteristics of a given location, not the weather on any specific day. Exploratory data analysis indicated that some WorldClim variables introduced spatial mapping artifacts into model predictions. Therefore, the present invention includes three WorldClim bioclimatic variables as covariates: BIO1 (mean annual temperature), BIO16 (precipitation of the wettest quarter), and BIO17 (precipitation of the driest quarter).

Summaries of gridded daily records are generated from the NCEP CFS version 1. The NCEP CFS data product is a 6 hourly summary of weather-related variables at 0.3-degree resolution (about 30 km). The variables are precipitation, soil moisture, water runoff, minimum temperature, maximum temperature, mean temperature, sensible heat net flux, downward shortwave radiation net flux, potential evapotranspiration, and transpiration. The 6-hour data product is aggregated within days using the arithmetic mean for soil moisture, mean temperature, potential evapotranspiration, transpiration, minimum temperature, maximum temperature, sensible heat net flux and downward shortwave radiation net flux, and the sum for precipitation and water runoff. Six-month and three-year summaries were generated for each variable using the arithmetic mean for the corresponding time interval prior to the target date. The target date is the date of collection of an in-situ soil sample or the date for which a prediction is desired. The NCEP data record is not available prior to April 2011. All NCEP summaries are treated as missing observations for records associated with sample dates that precede the NCEP data record (604 or 605 records, depending on the NCEP variable).

Although the NCEP summaries provide gridded temperature data at 0.3-degree resolution, variation at finer resolution influences biogeochemical processes and SOC. NCEP summaries are supplemented with the 1 km MODIS land surface temperature and emissivity data product. MOD11A2 is an 8-day composite with 1 km native resolution. For each 1 km pixel in the MOD11A2 V006 data set, the arithmetic mean is computed in the six-month interval prior to the target date.

In accordance with the present invention, surface elevation is derived from the USGS 3DEP 10 m digital elevation model. Six-month and three-year soil-moisture summaries are derived from the SMAP L3 daily 9 km soil moisture data product. Summaries were the arithmetic mean within six-month and three-year windows prior to the target date. The covariate data set contained estimates of soil properties from the SoilGrids version 2.0 global 250 m data product. Gridded clay, sand and silt content as covariates is preferably used.

Sentinel-1 measurements derived from Level-1 Ground Range Detected data products are preferably used. Sentinel-1 is a C-band radar that acquires images in all weather conditions at a variety of spatial resolutions. Images acquired with dual polarizations (VH and VV) in the Interferometric Wideswath (IW) mode from ascending orbits are preferably used. For each 20 m pixel in the Sentinel-1A data set, we computed the median within a six-month window prior to the target date.

Remote sensing summaries are used from Sentinel-2 bottom of atmosphere surface reflectance data products. For a given location there is a set of candidate Sentinel-2 observations within a time interval that could be used for training and prediction. We refer to the problem of selecting and summarizing this set of observations as image reduction, and we refer to the derived summaries as features. Engineering an image reducer to generate features requires selecting three criteria: the time interval over which the reducer will be applied, filtering that determines whether a candidate observation is included in the summary and choosing the reducing function. For example, a cloud shadow and cloud cover filtering criteria is preferably used to time series Landsat observations over the 2000-2012 time interval to generate a global map of forest cover change. Filtered time series Sentinel-2 surface reflectance uses the NBR2 and NDVI to generate a bare soil composite. For data-driven prediction, where a model from training data is applied in the real world, the reducer must produce summaries that are spatially and temporally consistent with minimal spatial mapping artifacts. Spatial mapping artifacts can occur when there are spatially structured errors in covariate features that propagate through model predictions.

Individual bands and derived indices from Sentinel-2 level 2 surface reflectance data products are used according to the invention to derive features from optical remote sensing using two image reducers. These two image reducers allow the ATLAS-SOC algorithm to use covariates that represent different intervals of time. The first of these, which we call the simple reducer, computes the arithmetic median over all candidate observations for every individual location within the six-month window prior to the target date. The simple reducer thus considers the relationship between image features and SOC immediately prior to the SOC measurement. For example, if an in-situ soil sample was collected on Apr. 9, 2021, the simple reducer would use Oct. 9, 2020-Apr. 9, 2021. If a prediction is desired using a calendar date of Oct. 16, 2021, the simple reducer would use Apr. 16, 2021-Oct. 16, 2021. The second version, which we call the time-series summary reducer, computes the arithmetic median within sequential, three-month time windows over the previous 24 months. The time-series summary reducer generates statistical summaries of the land surface that are related to agricultural practices over the previous 24 months, including planting and harvest time, cover cropping, and tillage status.

Indices are selected that track green and senescent vegetation cover, tillage status, and the presence of water on the land surface. These indices were selected because they are likely to be associated with land-surface variables associated with the amount of carbon in soil and how it is changing over time. Below we define each index. Band definitions for Sentinel-2 are blue (B2; 460-530 nm), green (B3; 540-0.580 nm), red (B4; 650-680 nm), near-infrared (B8; 780-890 nm), SWIR1 (B11; 1,570-1,660 nm), and SWIR2 (B12; 2,110-2,290 nm).

NDVI is then correlated with cover of green vegetation and therefore distinguishes vegetation from bare-soil conditions. NDVI is computed using the simple reducer and time-series summary reducer according to:

$$NDVI = \frac{NIR - red}{NIR + red}$$

The SAVI is a modification of NDVI that corrects the index in the presence of minimal vegetation cover. SAVI is positively associated with vegetation cover. We computed SAVI using the simple reducer according to:

$$SAVI = 1.5 \times \frac{NIR - red}{NIR + red + 0.5}$$

A known SATVI is also used. The index is positively associated with green and dry vegetation. SATVI was calculated using the simple reducer according to:

$$SATVI = 1.5 \times \frac{SWIR 1 - red}{SWIR 1 + red + 0.5} - \frac{SWIR 2}{2}$$

The BSI is used to identify pixels with minimal vegetation cover and exposed soil. BSI has been used to develop bare-soil composites of agricultural land using blue, red, and shortwave-infrared reflectance. BSI is negatively associated with vegetation cover. BSI is computed using the simple reducer and time-series summary reducer according to:

$$BSI = \frac{( red + SWIR 1 ) - ( NIR + blue )}{( red + SWIR 1 ) + ( NIR + blue )}$$

The invention also uses the known NBR2 index to generate bare soil composites for prediction of organic carbon content using Sentinel-2. Larger values of NBR2 are typically associated with moist soils or crop residues. The NBR2 index was calculated using the simple reducer according to:

$$NBR 2 = \frac{NIR - SWIR 2}{NIR + SWIR 2}$$

The known NDTI takes advantage of changes in shortwave infrared reflectance in water absorption regions to detect surface roughness associated with tillage status. Larger values of the index are more likely to be associated with conventional tillage status. We computed NDTI using the simple reducer according to:

$$NDTI = \frac{SWIR 1 - SWIR 2}{SWIR 1 + SWIR 2}$$

The known BI is associated with total brightness. Under bare soil conditions, brighter soils have less organic matter content than darker soils do. The BI was calculated using the simple reducer,

$$BI = \sqrt{\frac{{red}^{2} + {green}^{2}}{2}}$$

The known LSWI is positively correlated with the total content of liquid water in vegetation and soil. It was calculated using the simple reducer according to:

$$LSWI = \frac{NIR - SWIR 1}{NIR + SWIR 1}$$

Tasseled cap brightness, greenness and wetness is computed using the Sentinel-2 coefficients. Tasseled cap brightness was calculated using the simple reducer according to:

$$brightness = \text{?} \times blue + \text{?} \times green + \text{?} \times red + \text{?} \times NIR + \text{?} \times SWIR 1 + \text{?} \times SWIR 2$$

$$\text{?} \text{indicates text missing or illegible when filed}$$

Tasseled cap greenness was calculated using the simple reducer according to:

$$greenness = \text{?} \times blue - \text{?} \times green - \text{?} \times red + \text{?} \times NIR + \text{?} \times SWIR 1 + \text{?} \times SWIR 2$$

$$\text{?} \text{indicates text missing or illegible when filed}$$

Tasseled cap wetness was calculated using the simple reducer according to:

$$wetness = \text{?} \times blue + \text{?} \times green + \text{?} \times red + \text{?} \times NIR - \text{?} \times SWIR 1 - \text{?} \times SWIR 2$$

$$\text{?} \text{indicates text missing or illegible when filed}$$

The algorithm of the present is a weighted gradient-boosted regression tree XGBoost. XGBoost is used because tree-based regression consistently performs well on a wide-range of prediction tasks using tabular data, including SOC mapping. Off-the-shelf implementations are efficient to train, able to handle missing values, and produce interpretable feature-importance metrics. The XGBoost algorithm works by assembling an ensemble of weak learners corrected iteratively to reduce bias and variance. Every weak learner is a single regression tree developed using recursive binary splitting and a user-defined objective function. Here the objective function was the mean absolute error:

$$MAE = \frac{1}{n} {\sum }_{i = 1}^{n} abs ( {y}_{i} - {\hat{y}}_{i} )$$

where n, is the number of training samples in the algorithm, yᵢ is measured SOC (%) for sample i, and ŷᵢ is the predicted value of SOC (%) for sample i.

In accordance with the present invention, the model was fitted to all 5,230 in-situ soil samples and their associated covariates. The 4,260 data records collected in CONUS agriculture were weighted equally with a value of 1. For the remaining 970 measurements collected under the USDA RaCA program, we explored weights of 0-1 in increments of 0.05 and examined cross-validated model performance under each weighting scenario. The purpose of down weighting RaCA samples was to allow previously collected data to contribute broad spatial coverage while maintaining the importance of recently collected data in agricultural land and during the period of Sentinel-2. The ability of the model is evaluated to predict SOC (%) associated with individual in-situ soil samples and aggregated field means. Because the model was trained using physical samples, the physical-sample performance is simply the geographically-dependent cross-validated regression described below. To evaluate the performance of the model at the field level, the mean SOC (%) is computed using in-situ soil samples in fields that contained ≥5 samples (n=165 fields and 3,285 in-situ soil samples) and the associated mean of predicted values at the same sample locations in each field.

When models are trained using a nonrandom sample of the prediction domain, cross-validation should use geographically dependent partitions. The idea is to understand how the algorithm will perform when transferred to a geographic subset of the prediction domain that is different from training data. Individual units of land management (agricultural fields) are used as cross-validation folds. Most samples in the invention's data collected under the USDA RaCA program are spatially isolated (mean nearest-neighbor distance=30.6 km, range=0.3-198.1 km). In contrast, new measurements collected in 2020 and 2021 were acquired on individual fields with multiple in-situ soil samples (mean=10.4 samples per field, range=3-133).

Model performance is then evaluated using the intercept and slope of the cross-validated linear regression, the RRMSE, and the coefficient of determination (R²). The RRMSE was computed using:

$$RRMSE = \frac{\sqrt{\frac{1}{n} {\sum }_{i = 1}^{n} {( {y}_{i} - {\hat{y}}_{i} )}^{2}}}{\frac{1}{n} {\sum }_{i = 1}^{n} {y}_{i}} \qquad ( 13 )$$

where yᵢ indicates a measurement of SOC (%) using physical soil sampling, and ŷᵢ is the predicted value derived from machine-learning. The coefficient of determination was computed according to:

$${R}^{2} = \frac{{SS}_{total} - {SS}_{residual}}{{SS}_{total}} \qquad ( 14 )$$

where SSₜₒₜₐₗ and SS_{residual} are the total and residual sums of squares from the cross-validated regression, respectively. Because SOC (%) is bounded at 0 and 100, it is assumed a truncated Gaussian error term when computing the cross-validated regression. It is quantified whether model performance varied with depth of the soil measurement using indicator-variables regression. The regression model is:

$${y}_{i} = {b}_{0} + {b}_{1} {X}_{1} + {b}_{2} {X}_{2} + {b}_{2} {X}_{1} {X}_{2} + {∈}_{i} \qquad ( 15 )$$

where X₁ is the predicted value derived from machine-learning in units of percentage-by-mass and X₂ is binary indicator variable that dictates whether YF was measured over the 0-5 (X₂=0) or 0-30 (X₂=1) cm depth range. The parameter E is a Gaussian error truncated over the range 0-100. When the value of X₂=0, the equation collapses to the ordinary linear regression on samples over the 0-5 cm depth range. When X₂=1, b₂ and b₃ test the null hypothesis that the cross-validated intercept and slope are equivalent between 0-5 and 0-30 cm depth ranges, respectively. The parameters b₂ and b₃ are the change in the intercept and slope for the case where X₂=1.

XGBoost supports numerous hyperparameters that can be defined prior to fitting the model. These hyperparameters can be assigned default values or optimized in a process called hyperparameter tuning. Here, five hyperparameters are tuned: the number of trees in the ensemble, the learning rate, the proportion of columns used to identify the split at each node, the proportion of samples used by each tree, and the maximum depth of each tree. Bayesian optimization is used to identify hyperparameter values. Values are selected that resulted in the smallest sample-level mean absolute error using the cross-validation described above.

An objective of the method and system of the present invention is to produce a DSM algorithm that can predict SOC as a percentage by mass in North American agricultural land. However, it is also needed to determine whether existing data products accurately estimate field-level SOC as a percentage by mass. Although existing publicly available maps of SOC that include CONUS agricultural land were not designed for agricultural land in particular, they might be useful to carbon market programs for establishing baselines in carbon removal projects, or for initialization of biogeochemical simulations of SOC fluxes. However, the accuracy of these data products in agricultural land in particular has not been assessed. These data products are generally developed using legacy soil sample data. Validating them against recent soil samples provides a useful assessment of their real-world applicability in current agricultural conditions. Four publicly available SOC data products were compared to independent measurements of SOC as a percentage by mass.

Further expanding on the discussion above, SoilGrids version 2.0 is a global 250 m data product that contains estimates of SOC (%) at multiple soil depths. The SoilGrids 2.0 data product was developed using a QRF algorithm trained on about 240,000 observations worldwide and >400 environmental covariates. Covariates included climate and temperature, geology and landcover, vegetation indices and raw measurements from the Landsat and MODIS sensors. The SoilGrids 2.0 data set contains predictions of SOC and other properties within six depth intervals. Predictions from 0-5, 5-15 and 15-30 cm beneath the soil surface are used to derive an estimate of SOC within the top 30 cm of soil. This is achieved by computing a weighted average, where the weight applied to each depth interval was proportional to the range of that interval over 0-30 cm. For example, the weights applied to 0-5, 5-15 and 15-30 cm were ⅙, ⅓, and ½.

In accordance with the invention, 100 m resolution soil property and class maps are generated using an approach that differs by integrating multiple soil data sets within a common analytical framework and by predicting SOC at specific depths beneath the soil surface, rather than over depth ranges. Soil data sets include the National Cooperative Soil Survey Characterization Database, the National Soil Information System, and measurements from the USDA RaCA program. Linear interpolation are used among SOC predictions at 0 cm (surface), 5 cm, 15 cm and 30 cm to produce an SOC profile with 1 cm increments over the 0-30 cm depth range. Then, the unweighted arithmetic average is computed over all 30 increments to produce a single estimate for the top 30 cm of soil in every 100 m pixel.

The SSURGO database has been remapped at 30 m resolution using machine learning and environmental covariates (POLARIS). The POLARIS data product does not contain estimates of SOC (%). POLARIS estimates of organic matter are converted according to the invention within three depth ranges to SOC as a percentage by mass using the van Bemmelen factor of 0.58. Values other than 0.58 were considered to determine whether alignment between POLARIS data product and in-situ soil samples was sensitive to the conversion factor used.

The van Bemmelen factor can be used to convert organic matter into units of SOC according to the following:

$$SOC = SOM \times 0.58$$

where SOC and SOM are soil organic carbon (%) and soil organic matter (%), and 0.58 is the van Bemmelen factor. The value of the conversion factor has been questioned, with alternative values in the range of 0.4-0.71. The van Bemmelen factor of 0.58 is used to express POLARIS estimates of SOM in units of SOC (%) so that they could be validated against independent SOC measurements obtained using dry combustion.

Alternative conversion factors were applied over the interval 0.01-2 in increments of 0.01. For each value we computed the MAE and the linear regression relationship between measured values of SOC (%) as the response variable and converted values from POLARIS as the independent variable. It is not asserted that this conversion results in a physically or biologically meaningful interpretation of the POLARIS data product, but this analysis is used to understand whether any rescaling of the POLARIS data product results in a better validation against independent measurements.

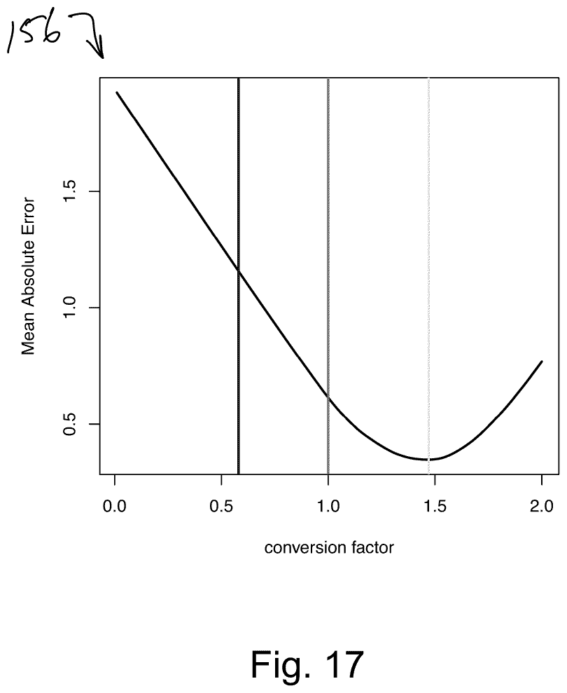

The relationship between MAE and the conversion factor is shown as 156 in FIG. 17. A conversion factor of 1.47 minimizes the MAE. The intercept and slope of this relationship are −0.409 and 1.185, respectively. A conversion factor of 1 results in a smaller value for the MAE than the van Bemmelen factor of 0.58 (i.e. the correspondence between POLARIS SOM and measured SOC is stronger than the correspondence between POLARIS SOC and measured SOC).

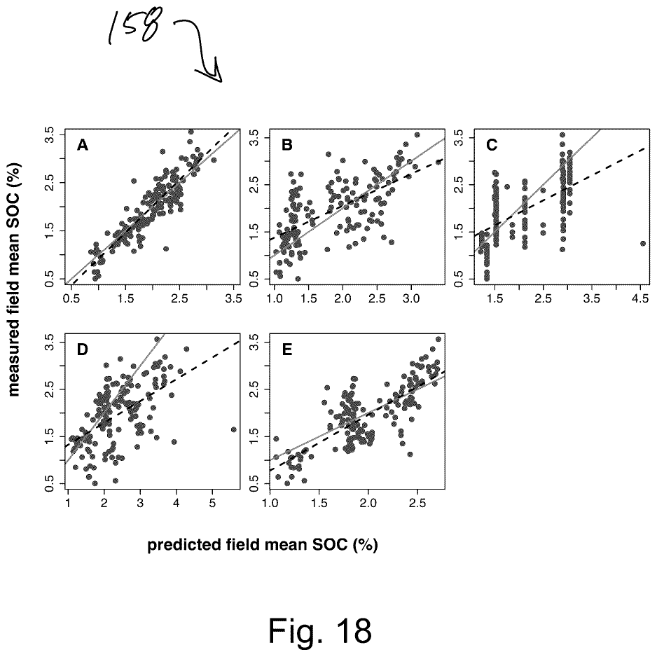

The value of the conversion factor that minimizes the MAE is used to generate FIG. 18. FIG. 18 shows points are 165 fields with ≥5 samples (total number of samples=3,285). (A) ATLAS-SOC. (B) A publicly available 100 m soil properties and class map. (C) the Harmonized World Soil Database version 2.0. (D) SoilGrids version 2.0. (E) POLARIS (based on a conversion factor of 1.47). The solid line is the one-to-one relationship, and the dashed line is the best-fit linear regression.

See FIG. 17 which shows MAE between field mean SOC and converted POLARIS SOM for values of the conversion factor between 0.01 and 2 in increments of 0.01. The left vertical line is the van Bemmelen factor, the middle vertical line has a value of 1 (no conversion), and the right vertical line is the value that minimizes the MAE (1.47).

Predictions from 0-5, 5-15 and 15-30 cm beneath the soil surface are used to derive an estimate of SOC (%) within the top 30 cm of soil using the same weighted average procedure applied to the SoilGrids version 2.0 data product described above.

The HWSD2 is a 2023 compilation of national soil surveys and legacy maps distributed as a global 30-arc-second raster (about 1 km resolution). Each pixel (called a soil mapping unit in HWSD2) is linked to attribute tables that report soil properties for seven depth layers. Because a given mapping unit can contain >1 soil unit, each of which has distinct properties, we first calculated area-weighted SOC (%) for each mapping unit. The area weights for each mapping unit were based on the SHARE percentage, which is the area of each soil mapping unit contained within each soil unit. Negative values in the SOC variable were ignored in subsequent analysis. To estimate mean SOC (%) in the top 30 cm, we combined the 0-20 cm layer (D1) with half of the 20-40 cm layer (D2) using weights of ⅔ and ⅓, respectively.

Also, the field mean SOC (%) is calculated over the 0-30 cm depth range using each publicly available SOC data product within each of 165 fields with ≥5 in-situ soil samples. This is achieved by re-projecting field polygons from the World Geodetic System 1984 coordinate reference system (EPSG:4326) to the native projection associated with each data product. For each of the four data sets, pixels are extracted that intersected the field geometry. Because some pixels were on boundary edges, the proportion, wᵢ, of each pixel, xᵢ, was calculated that was contained within field j, and used these proportions to compute the weighted mean SOCⱼ as a percentage by mass according to,

$${SOC}_{j} = \frac{{\sum }_{i = 1}^{n} {w}_{i} {x}_{i}}{{\sum }_{i = 1}^{n} {w}_{i}}$$

In accordance with the present invention and the calculations discussed herein in detail, SOC can be predicted with a high degree of precision and accuracy in North American agricultural land using machine learning. Analysis of the indicator-variables regression showed that model performance was independent of the measured depth range. At the sample level, parameter estimates were b₀=−0.199 (P=0.959), b₁=1109 (P=0.028), b₂=−0.068, (P=0.978), and b₃=0.010 (P=0.994). Therefore, the indicator variable is dropped and fit the linear relationship between measured and predicted values. The intercept and slope of the cross-validated regression without indicator variables were b₀=−0.249 (P<0.001) b₁=1.130 (P<01,001), respectively.

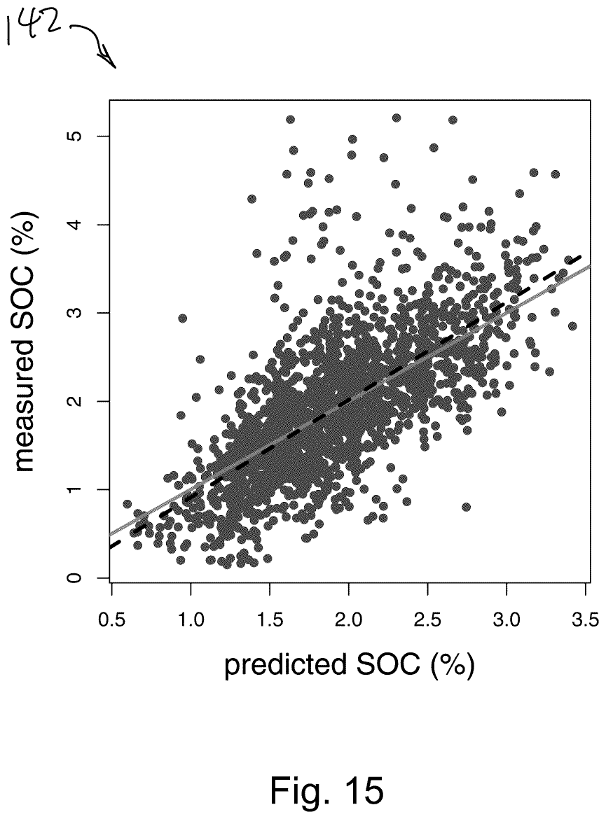

Turning back to FIG. 15, the coefficient of determination was R²=0.487 and RRMSE, was 0.168. The grey line is the 1:1 relationship, and the dashed line is the best-fit linear regression. This plot shows a random sample of 2,000 points for clarity, but the full data set contains 5,230 observations.

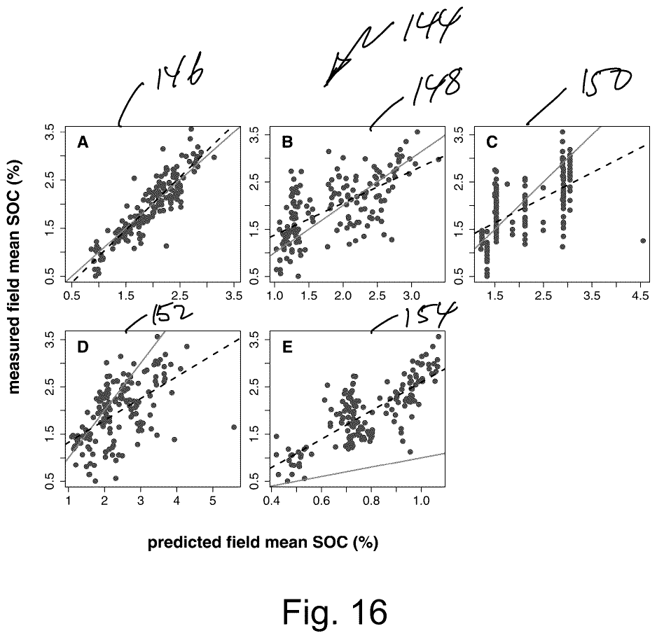

FIG. 16 shows aggregation to the field level demonstrated that mean predictions are accurate for five samples A-E 146, 148, 150, 152, 154, which illustrates relationships between field mean SOC (%) and predicted values. Points are 165 fields with ≥5 samples (total number of samples=3,285). (A) ATLAS-SOC. (B) A publicly available 100 m soil properties and class map. (C) the Harmonized World Soil Database version 2.0. (D) SoilGrids version 2.0. (E) POLARIS (based on the van Bemmelen factor). The solid line is the one-to-one relationship, and the dashed line is the best-fit linear regression.

Field-level predictions are evaluated by computing the arithmetic mean in fields with ≥5 in-situ soil samples (n=165 fields, 3,285 in-situ soil samples). The observed value (response variable) was the mean from sampled soil cores in the field, and the predicted value (independent variable) was the mean from pixel-level predictions at the same locations in each field. Predicted field means were always from an instance of the geographically dependent cross validation that did not contain any in-situ soil samples from the observed field. The intercept and slope of the cross-validated relationship were b₀=−0.179 (P=0.333) and b₁=1.095 (P<0.001). The coefficient of determination was R²=0.811, and the RRMSE was 0.041.

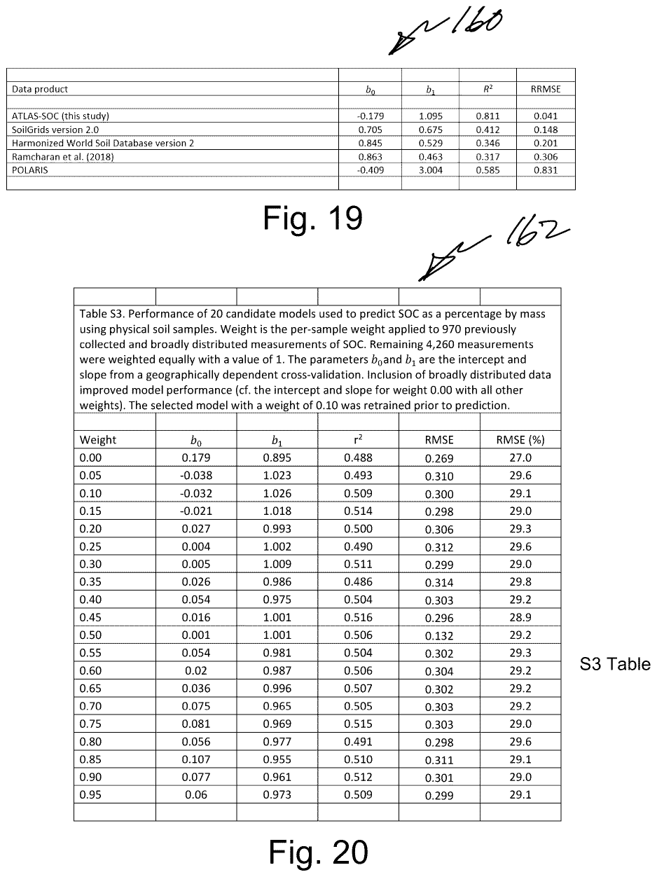

Turning now to FIG. 19, a table 160 is provided showing a comparison of field measurements of SOC (%) to four publicly available SOC data products. Publicly available data products showed poor ability to reproduce measured values. Ranked by RRMSE, the best-performing publicly available data product was the SoilGrids version 2.0 dataset, followed by HWSD2, the 100 m resolution soil property and class map and POLARIS. All of these data products exhibited substantial bias in comparison to measured values, and none of them out-performed ATLAS-SOC, as also shown in FIGS. 16 and 18. More specifically, FIG. 19 shows a table that is a validation of mean SOC (%) in 165 agricultural fields measured using 3,285 in-situ soil samples. b₀ and b₁ are the intercept and slope of the linear regression relating mean SOC from in-situ soil samples (response) to the predicted value from four publicly available data products and ATLAS-SOC.

The SoilGrids version 2.0 data product, HWSD2, and the 100 m soil property and class map underestimated field measurements at values <2.17%, 1.79%, and 1.61%, respectively, and overestimated field measurements at other values. The POLARIS dataset underestimated the field mean SOC (%) in all 165 fields, although the degree of correspondence improved when a different conversion factor was used, as in FIG. 18.

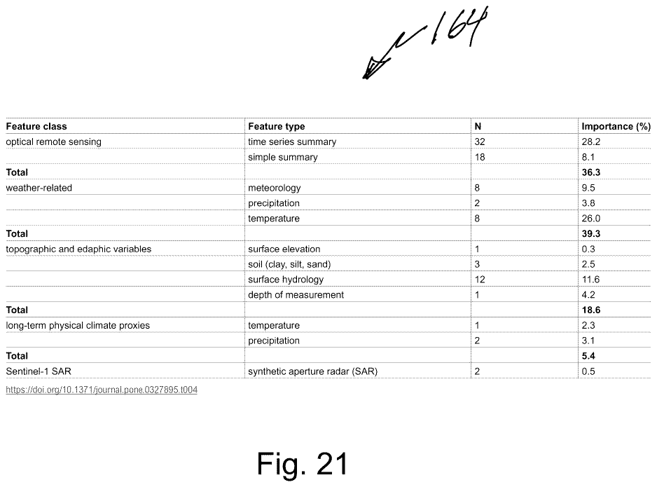

Feature importance scores were computed for every covariate in the training set. The top 20 features accounted for 68.6% of the overall importance in the data and included 7 variables from optical remote sensing, 8 weather-related proxies, 2 topographic and edaphic variables, and 2 climate variables. The most important feature type was Sentinel-2 optical remote sensing time series summaries, which collectively accounted for 28.2% of the importance in the data, followed by weather-related temperature (26.0%) and surface hydrology (11.6%), as seen in FIG. 21, which shows relative importance for 90 features used to predict SOC (%) in CONUS agricultural soils. Importance is from a gradient-boosted regression fitted to 5,230 measurements of SOC linked to covariate features and summed within feature types. For example, there are 32 time-series features from Sentinel-2 optical remote sensing whose aggregate importance is 28.2%. Totals are marginal sums within feature classes.

Among feature classes, weather-related variables contributed 39.3% of the importance in the data, most of which was due to temperature (26.0%), followed by meteorology (9.5%) and precipitation (3.8%). Optical remote sensing summaries and their derivatives collectively contributed 36.3% of the importance in the data. Time series optical remote sensing summaries and their derivatives (28.2%) were about 3.5 times more important than summaries generated using the simple reducer (8.1%). Topographic and edaphic features were responsible for 18.6% of overall variable importance. Features related to surface hydrology were the most important topographic and edaphic features (11.6%), followed by the depth of the soil measurement (4.2%), silt, sand, and clay content (2.5%) and surface elevation (0.3%). Long-term physical climate proxies accounted for 5.4% of the importance in the data. FIG. 21 also shows two measurements from Sentinel-1 SAR that were relatively unimportant, contributing 0.5% to the overall importance in the data set.

Carbon market programs in agriculture require accurate measurement techniques that can be deployed efficiently at scale. Our analysis shows that data-driven, DSM methods can accurately predict mean SOC (%) in CONUS agricultural land. This analysis is based on 5,230 in-situ soil samples collected in actively cultivated, conventional row-crop agriculture, natural prairies and grasslands within 47 US states. Using a gradient-boosted regression tree and geographically dependent cross-validation, a model driven was developed in accordance with the present invention by combinations of long-term climate proxies, topography and optical remote sensing variables that is both precise and accurate, overcoming challenges to widespread quantification that is necessary in support of carbon market programs.

Models to predict SOC are trained using measurements from individual soil cores matched to remote sensing pixels, but carbon market programs require estimates of SOC within regions. These regions could represent units of individual land management (agricultural fields) or aggregations of fields that total hundreds to thousands of hectares. Methodologies for carbon-credit generation penalize uncertainty in estimates of net GHG reductions at the scale of aggregation. Demonstrating that predictions are precise and accurate at the scale of aggregation is necessary to underpin rigorous carbon offsets in agricultural soil.

In accordance with the method and system of the present invention, the analysis, using a sample of 165 fields with ≥5 samples per field (n=3,285 samples in total), FIG. 16 shows that estimates of the field mean are precise and accurate when validated against fields that were excluded from model calibration. However, comparison of field measurements of SOC to four publicly available SOC data products demonstrated poor ability of other products to reproduce measured values. Validation statistics for these data products were worse than that reported in cases where summaries were available. This is because these data products were developed to perform well throughout large regions in general, not agricultural land in particular. These products were also developed using legacy soil sample data, and it is likely that agricultural and soil conditions have changed. The data archive used to train ATLAS-SOC for this analysis includes 5,230 in-situ soil samples that were collected in 47 US states and exclusively in agricultural conditions, most of which were collected recently. The limited ability of publicly available data products to generalize to agricultural land in our study is an example of Simpson's paradox, a problem in statistics where a relationship changes, or even reverses, in the presence of new variables. In the context of the carbon market programs, Simpson's paradox underscores the importance of model validation within the domain of application. It should not be assumed, for example, that a model with generally acceptable performance in North American soils will perform well in any given subset of North America that was poorly represented by calibration data.

The method and system of the present invention underscores the value of broadly distributed training data. Inclusion of geographically distributed in-situ soil samples collected under the USDA RaCA program decreased bias in comparison to a version where broadly distributed data were absent, as seen as 162 in FIG. 20. For example, when the model was trained exclusively on actively cultivated, conventional agriculture in the states of Arkansas, Colorado, Illinois, Iowa, Kansas, Minnesota, Nebraska, New Mexico, Oklahoma, South Dakota, Texas, and Wisconsin, it exhibited moderate bias, as indicated by the intercept and slope of cross-validated predictions (b₀=0.179, b₁=0.895). But when broadly distributed in-situ soil samples were added to the training set, bias reduced. Geographically distributed in-situ soil samples reduced bias in the model even though they accounted for <20% of the training set, and even though optical remote sensing and some weather variables were treated as missing values in broadly distributed data. This finding suggests that the value of geographically distributed in-situ soil samples in our analysis was primarily their contribution to resolving the relationship between SOC (%), long-term physical climate proxies, and topographic and edaphic variables. This indicates that when combined with contemporary in-situ soil data, legacy soil samples can play an important role in DSM efforts.

The results of the present invention illustrates that optical remote sensing is an important and independent source of information about SOC (%) in soils. Other data-driven approaches to SOC quantification indicate that combinations of physical climate and topography are the strongest predictors of SOC in soils of North America and China. Features from optical remote sensing are typically less important. The ranking of optical remote sensing in aggregate in two previous studies placed them at rank 3 or 4 out of 5 feature sets. However, weather-related variables and optical remote sensing summaries contributed 39.3% and 36.3% of the overall importance in the data, and 8 of the top 20 individual features were derived from optical remote sensing summaries, as 164 in FIG. 21.

Time series summaries from Sentinel-2 were the strongest optical remote sensing predictors of SOC (%), as in FIG. 21. In particular, median NDVI within three-month windows during the previous two years represented 5 of the top 20 most important features. Overall, time series summaries represented 28.2% of the importance in the data, a number 3.5 times larger than static summaries that do not consider changes in land cover over time. The sensitivity of remote sensing summaries from specific times of year suggests that land use and land cover changes are responsible for the strength of these summaries in comparison to other descriptors. One hypothesis for the importance of optical remote sensing variables in our analysis in comparison to previous work is that we increased the number of remote sensing features in comparison to other variables and used time-series feature engineering to generate predictors that are likely to be correlated with SOC (%). Point-in-time optical remote sensing measurements may be less strongly correlated with SOC (%) than summaries that target specific land management actions.

The ranking of optical remote sensing variables in a DSM framework to quantify SOC (%) is important, because optical remote sensing variables are a source of information about land management, including cover cropping and reduced or no-tillage practices that are expected to impact SOC sequestration. A data-driven framework that is sensitive to climate, weather, and topographic and edaphic conditions, but insensitive to variables related to land management will be able to recapitulate known physical gradients but may not detect changes related to farm practice. More work is needed to determine whether the inclusion of practice data or biogeochemical variables in a covariate feature set, such as cover-cropping and tillage status or variables related to microbial activity, improves performance, whether data-driven calculations are sensitive to those variables, and whether direct inclusion of these variables is redundant with optical remote sensing summaries.

Interpretation of feature importance can be influenced by the number and type of variables within a covariate set and how the feature summary is generated. Our analysis contained 50 optical remote sensing features and 18 weather-related variables, 17 topographic and edaphic variables, 3 long-term physical climate proxies, and 2 measurements from Sentinel-1 SAR. This indicates that the algorithm is being presented with more opportunities to model the relationship using optical remote sensing than other variable types, and that overall variable importance within categories can be influenced by imbalance in the feature set. Imbalance in a covariate feature set can be desirable when an objective is to ensure sensitivity to particular kinds of features that are overrepresented.

The approach developed in accordance with the present invention overcome the challenges of efficient quantification and scale in carbon market programs within agricultural soils. This is far superior to prior art methods and systems. The present invention can be contrasted with two alternative methods, physical soil sampling and biogeochemical simulation. Physical soil sampling produces locally accurate measurements of SOC, but these measurements are costly to acquire and may demand unreasonably large sample sizes to generate precise area-based summaries required by carbon market programs.

Also, biogeochemical simulations stand on decades of research in biogeochemistry and soil science. They use local information about weather, climate, soil characteristics and farm management data to simulate interactions in soil using physics and chemistry. For example, the DeNitrification-DeComposition biogeochemical simulation uses climate, soil and cropping variables. Some of these variables must be supplied by the grower (e.g., the fraction of crop residue left as stubble in the field after harvest) and other variables are difficult to measure at scale (e.g., the background NH₃ concentration in the air). Data-driven methods from the field of DSM can help to reduce barriers to large-area estimation by allowing machine learning to model relationships between input variables and SOC and acquiring input variables from publicly available sources.

Therefore, data-driven calculation of SOC in agricultural soil is achievable at scale using DSM methods that are sensitive to land use and land cover change of the present invention. The ATLAS-SOC algorithm developed here can predict SOC as a percentage by mass with errors that are small when evaluated at the physical sample and field mean levels. This algorithm was trained using a data set that explicitly represented agricultural land in CONUS. Field-level validation of four publicly available data products in CONUS demonstrated poor ability of publicly available SOC data products to reproduce independently measured values from in-situ soil samples.

In sum, the present invention improves on prior art by employing three-part statistical validation (coverage, fit, bias), geographically dependent or random cross-validation, multi-scale validation (pixel, field, project), explicit treatment of spatial autocorrelation, and explicit treatment of temporal covariance.

As to data processing, the prior art merely uses static feature generation, single-source satellite data, manual quality control, and fixed temporal windows, the method and system of the present invention improves on such data processing by using instead versioned feature store architecture, multi-sensor data fusion, automated quality filtering, and dynamic temporal aggregation.

Therefore, the present invention provides an improved method and system for measuring SOC that is more accurate, more scalable and less expensive than prior art methods and systems for such SOC measurement. The present invention combines remote sensing, soil samples, machine learning, and biogeochemical principles in a new and novel way to achieve highly accurate SOC quantification while dramatically reducing the need for physical soil sampling. Most notably, the correlation between SOC and BD set forth herein is a significant advance over prior art methods and systems. The present invention provides a method and system that is implemented in a new and novel way that is not found in the prior art.

The aforesaid examples are only one of the potentially many applications and modes of execution of the system of the present invention and common changes and substitutes made by technical personnel of this field within the technical proposal of this invention should be included in the protection scope thereof. It would be appreciated by those skilled in the art that various changes and modifications can be made to the illustrated embodiments without departing from the spirit of the present invention. All such modifications and changes are intended to be covered by the appended claims.

What is claimed is:

1. A method for quantifying SOC present in a landscape and determining the uncertainty thereof, comprising the steps of:
obtaining remote sensing data;
extracting biogeochemical information from the remote sensing data;
collecting environmental data, including physical climate proxies, weather related variables, topographic and edaphic measurements and other measurements;
collecting soil core samples of the landscape;
providing a validation framework;
quantifying SOC present in the landscape by combining the remote sensing data, environmental data, soil samples, the validation framework test, and the biogeochemical information to quantify SOC present in the landscape;
estimating uncertainty at a point of level for SOC and BD using conformal predictions and QRF; and generating statistically valid prediction intervals for each pixel-level estimate.

2. The method of claim 1, further comprising the step of:
aggregating uncertainties across space using geostatistical techniques to account for spatial autocorrelation to ensure that uncertainty estimates remain valid when scaling from individual points to field and project levels.

3. The method of claim 2, wherein the spatial autocorrelation employs variogram analysis.

4. The method of claim 1, wherein the step of estimating uncertainty includes carrying out a cross-scale uncertainty propagation methodology.

5. The method of claim 1, wherein the step of estimating uncertainty includes temporal covariance incorporation.

6. The method of claim 1, wherein the validation framework includes conducting validation tests with statistical validation with coverage, fit, and bias.

7. The method of claim 1, wherein the validation framework includes a cross-validation.

8. The method of claim 1, wherein the validation framework includes multi-scale validation with pixel, field and project.

9. The method claim 1, wherein data in the validation framework uses multi-sensor data fusion.

10. The method of claim 1, wherein data in the validation framework uses temporal aggregation.

11. The method of claim 1, wherein processing of the remote sensing data includes remote sensing variables to map SOC.

12. The method of claim 1, wherein SOC stock is determined by SOC Stock=SOC %×BD×depth.
